(19) United States
(12) Patent Application Publication
(10) Pub. No.: US 2004/0076590 A1
(43) Pub. Date: Apr. 22, 2004

(54) Antibacterial toothpaste and mouthwash formulations

(76) Inventor: Joe S. Wilkins JR., Galveston, TX (US)

Correspondence Address: Laura G. Barrow, Esq., P.O. Box 215, Estero, FL, 33928-0215, US

(21) Appl. No.: 10/615,588

(22) Filed: Jul. 8, 2003

Related U.S. Application Data
(60) Provisional application No. 60/394,333, filed on Jul. 8, 2002.

(51) Int. Cl. A61K 7/16

(52) U.S. Cl. 424/49

(57) ABSTRACT

Novel toothpaste and mouthwash formulations are described herein. The formulations comprise therapeutically effective amounts of d-limonene for inhibiting the growth of or eradicating pathogens found in the oral cavity of animals.

This is application claims the benefit of the filing of co-pending U.S. provisional application serial No. 60/394,333 filed Jul. 8, 2002, which is incorporated by reference herein in its entirety.

### BACKGROUND AND SUMMARY OF THE INVENTION

Limonene is a monocyclic monoterpene commonly found in the form of its d-isomer. d-limonene is one of the most common terpenes in nature, occurring in citrus and a wide variety of other plant species.

The present invention is directed to toothpaste and mouthwash formulations. In particular, the formulations comprise, in part, limonene as an active ingredient in killing or inhibiting the growth a variety of bacterial pathogens known to cause a number of infectious diseases in humans and animals. Specifically, in vitro analyses revealed that d-limonene is effective in eradicating the following major gram-positive pathogens: Staphylococcus aureus, Staphylococcus epidermidis (both methicillin sensitive and resistant), Streptococcus pyogenes, Streptococcus mutans, and other beta hemolytic streptococci, Entercoccus faecalis, and Enterococcus faecium (both vancomycin sensitive and resistant). In vitro tests further revealed that d-limonene is effective in eradicating the following gram-negative pathogens: Escherichia coli, Enterobactor cloacae, Klebsiella pneumoniae, Serratia marcescens, Pseudomonas aeruginosa, Acinetobacter baummii/haemolyticus, Paenibacillus polymyxa, and Stenotrophomonas maltophilia. In vitro tests also revealed that d-limonene is effective in eradicating various Bacillus species, such as Bacillus licheniformis, B. sphraericus, Bacillus cereus, and Bacillus subtilus, including the species strain of anthrax (Bacillus anthracis—Steams and Ames strains. The microbial assay methodology, and results, are described in Example 1 and Tables 1-2.

In view of the in vitro anti-microbial activity of d-limonene, the present invention is directed to formulations and methods of using these formulations for treating a variety of systemic and local bacterial infections in humans and animals, wherein an effective amount of d-limonene, preferably incorporated with one or more base components in a formulation, and then applied as a toothpaste or mouthwash to the human or animal. In particular, for localized infections within the mouth and throat (or for the prophylactic treatment thereof), the d-limonene may be formulated in a mouthwash that may be used as a rinse or a swab, for example. The d-limonene may also be formulated in a toothpaste, using excipients (i.e. base components) commonly employed in tooth paste formulations. If desired to aid in strengthening the teeth, calcium and/or magnesium compounds may be employed in these formulations, as well.

### DETAILED DESCRIPTION OF THE PREFERRED EMBODIMENTS

The present invention is directed to tooth paste formulations and mouthwash formulations for killing or inhibiting the growth a variety of bacterial pathogens known to cause a number of infectious diseases in humans and animals. As used herein, the term “animal” shall include humans as well as non-human animals, namely mammals and reptiles. Specifically, the present invention is directed to toothpaste and mouthwash formulations comprising limonene for use in killing or inhibiting the growth of common dental pathogens that are known to cause tooth decay and periodontal disease, such as Porphyromonas gingivalis, Bacteroides species, Actinobacillus action mycetemcomitons, Prevotella intermedia, Fusobacterium nucleatum, Bacteroides forsythus and other species, Campylobacter rectus, Eikenella corrodens, Peptostreptoloccus micros, Selenomonas sp., Eubacterium sp., Streptococcus intermedius, spirochetes Treponema denticola, and Treponema pallidum and syphillis. The inventive formulations are also useful in killing or inhibiting the growth of other pathogens that have been shown to colonize in the mouth and cause various systemic diseases, such as bacterial endocarditis and arthritis, or example.

The inventive toothpaste formulation has not only been shown to be effective in treating bleeding gums and receding gum lines, but it is effective in minimizing plaque buildup on the teeth to not only whiten the teeth, but minimize tooth decay.

In certain embodiments, the formulations comprise at least one base component and an active ingredient comprising limonene, preferably, a highly purified limonene (i.e. 98% and greater purity, more preferably about 98.5% to 99% purity). A preferred concentration range of limonene in the toothpaste formulations is from about 10% to about 40%. The d-limonene may be purified by known distillation techniques, such as that described in U.S. Pat. No. 6,420,435, which is incorporated herein by reference in its entirety.

The one or more base components employed in the tooth paste formulation include those typically found in conventional toothpastes, and thus the amounts and types of such base components are known by those of ordinary skill in the art. Exemplary base components include, but are not limited to, (a) sorbitol, a polyol which functions as a humectant/sweetener; (b) water, which functions as a diluent; (c) silica (e.g. ZEODENT, vended by Huber Corp.), which functions as an abrasive to help remove particles from the teeth; (d) glycerin, which also serves as a humectant; (e) surfactants, such as sodium lauryl sulfate or Polysorbate 20, for example; (f) binders and viscosity agents, such as CEKOL cellulose gum, xantham gum; and (g) preservatives, such as sodium benzoate and methyl parabens, for example. Flavoring and coloring agents (or whitening agents, like titanium dioxide) may be employed, as well. It will be appreciated by those of ordinary skill in the art that while the identified base components may indeed by employed in the present invention, other base components commonly employed in toothpaste formulations, now known or later discovered, may be used without departing from the scope and spirit of the present invention.

A preferred toothpaste formulation comprises from about 10% to about 40% d-limonene (98.0% or higher purity, more preferably 98.5%-99.0%); from about 15% to about 35% of sorbitol; from about 15% to about 30% of a silica agent (e.g. ZEODENT 113 and ZEODENT 165), from about 10% to about 20% water; from about 5% to about 15% glycerin, from about 2% to about 7% of surfactant (e.g. Polysorbate 20), from about 1% to about 2% flavoring agent (including sodium saccharin), from about 0.5% to about 1.5% of titanium dioxide, from about 0.5% to about 1.5% of binder (e.g. CEKOL 2000 gum), from about 0.05% to about 0.15% of a preservative (e.g. sodium benzoate), from about 0.25% to about 1.75% of pure calcium, and from about 0.10% to about 1.75% of magnesium phosphate.

The toothpaste formulation is particularly effective in improving receding and bleeding gum lines, which are typically caused by plaque and gingivitis as well as reducing dental decay.

Preferably, the toothpaste formulation further comprises a pharmaceutically acceptable calcium compound, preferably pure calcium and/or a pharmaceutically acceptable magnesium compound, such as magnesium phosphate, for promoting stronger teeth. Preferable tooth paste formulations comprise from about 18% to about 22% percent limonene. Preferable percentage amounts of calcium range from about 1.25% to about 1.50%. Preferable percentage amounts of magnesium phosphate range from about 1.25% to about 1.50%.

Preferred formulations for the inventive mouthwash effective in treating bacterial infections in the mouth (or inhibiting the growth of bacteria responsible for such infections) include an active ingredient comprising limonene, preferably a highly purified form of limonene (i.e 98.0% or greater purity, more preferably 98.5% to 99.0%) and one or more base components commonly employed in mouthwash formulations. Exemplary base components include (a) sorbitol; (b) polyethylene glycol (e.g. PEG 6) as a carrier and surfactant; (c) polysorbate (surfactant); (d) water (diluent); and (e) flavoring agents (e.g. sucralose). A preferred formulation comprises (a) from about 15% to about 25% of sorbitol, (b) from about 10% to about 20% of polyethylene glycol, (c) from about 2.5% to about 7.5% Polysorbate 20, (d) from about 2.5% to about 15% d-limonene, (e) from about 45% to about 65% water, (f) from about 0.2% to about 0.5% sucralose, and about 1.0% to 2.0% Belwood Wintergreen.

Administration of the inventive mouthwash is similar to conventional mouthwashes (i.e. about 30 ml placed within the mouth and swished about therein for about 30 seconds prior to expectoration); however, the administrated dose and time within the mouth may be varied as desired.

Notwithstanding the preferred toothpaste and mouthwash formulations described above, it is important to note that the d-limonene oil alone may be applied directly to the teeth or swabbed within the mouth, for example, for the purpose of killing or inhibiting the growth bacteria therein, although only small amounts of d-limonene should be used to prevent mucosal irritation that will result at higher amounts. It is also within the scope of the present invention to incorporate small amounts of pure d-limonene oil (e.g. about 0.1 ml) within a chewing gum base. Upon chewing of the gum, the d-limonene is released from the gum base and dispersed within the oral cavity and onto the teeth.

### EXAMPLE 1

Clinical isolates (10⁵ bacteria/ml) (about 100 μl) of gram-positive pathogens (Staphylococcus aureus and epidermidis (both methicillin-sensitive and resistant) plus Enterococcus faecalis and faecium) along with a group of gram-negative pathogens (Escherichia coli, Enterobacter cloacae, Klebsiella pneumoniae and Serratia marcescens coupled with opportunistic pathogens Pseudomonas aeruginosa, Acinetobacter baummii/haemolyticus and Stenotrophomonas maltophila) were each inoculated into 2 ml of d-limonene, in accordance with the standard phenol-coefficient assay and other screening methodology for plant antimicrobial activity and incubated for 72 hrs. A 2 ml broth media was used as a positive control. The d-limonene used was purified to at least 98.5% via a distillation process. The product was purified and examined for purity via HPLC.

Aliquots were subsequently cultured at 24 hours, 48 hours, and 72 hours to determine the antimicrobial effect. Appropriate media was inoculated in accordance with NCCLS standards. Blood agar was used for the gram-positive organisms, while McConkey Agar was utilized for the gram-negative organisms. ATCC strains of S. aureus, E. faecalis, P. aeruginosa, and E. coli were used as controls organisms and compared to the clinical isolates of these pathogens.

The results of the assay are shown in Table 1 (gram-positive organisms) and Table 2 (gram-negative organisms), wherein all of the pathogens tested were shown to be effectively eradicated within 24 hours.

Cultures were held 72 hours to ascertain if a resistant genetic code might have been facilitated. The response to subculture at 72 hours yielded no-growth, thus clearly indicating that no muta-genic or plasmid transposon was noted.

TABLE 1
Antibacterial effects of d-limonene on gram-positive organisms

| | Concentrations | | Growth | |
|---|---|---|---|---|
| Organism | CFU/ml | 24 hr | at 48 hr | 72 hr |
| S. aureus | >10⁵ | NG* | NG | NG |
| S. epidermidis | >10⁵ | NG | NG | NG |
| E. faecalis | >10⁵ | NG | NG | NG |
| E. faecium | >10⁵ | NG | NG | NG |

*NG = no growth

TABLE 2
Antibacterial effects of d-limonene on gram-negative organisms

| | Concentrations | | Growth | |
|---|---|---|---|---|
| Organism | CFU/ml | 24 hr | at 48 hr | 72 hr |
| E. coli | >10⁵ | NG | NG | NG |
| Ent. cloacae | >10⁵ | NG | NG | NG |
| K. pneumoniae | >10⁵ | NG | NG | NG |
| S. marcescens | >10⁵ | NG* | NG | NG |
| P. aeruginosa | >10⁵ | NG | NG | NG |
| Ac. baum/haemo | >10⁵ | NG | NG | NG |
| S. maltophilia | >10⁵ | NG | NG | NG |

*NG = no growth

### EXAMPLE 2

A toothpaste formulation was manufactured by combining the following components:

25.00% polyol (sorbitol)

20.00% Zeodent 113 (silica abasive)

20.000 d-limonene (at least 98.5% purity)

13.39% water

10.00% Glycerin Natural Kosher

5.00% Polysorbate 20

2.70% Zeodent 165 (silica abrasive)

1.00% Flavor 484 (Walmart brand)

1.00% titanium dioxide

1.00% CMC 9M31XF/Cekol 2000 (binder gum)

0.45% pure calcium

0.25% saccharin

0.11% magnesium phosphate

0.10% sodium benzoate

The foregoing components were combined as follows: the sodium saccharin and sodium benzoate were dissolved in the water and set aside. The Cekol and glycerin were combined, and, while mixing these two components together, the polyol was added. The solution of sodium saccharin and sodium benzoate were then added to the Cekol/glycerin, and polyol mixture. Next, Zeodent 165 was added to the mixture and blended in well, followed by the Zeodent 113, which in turn was blended in well until the mixture was free of lumps. Titanium dioxide, Polysorbate 20, and d-limonene were combined with the mixture and blended until the mixture was smooth. Finally, the calcium and magnesium phosphate was added, followed by the flavoring agent (i.e. Flavor 484).

### EXAMPLE 3

A toothpaste formulation was manufactured by combining the following components:

25.00% polyol (sorbitol)

18.00% Zeodent 113 (silica abasive)

20.000 d-limonene (at least 98.5% purity)

13.39% water

10.00% Glycerin Natural Kosher

5.00% Polysorbate 20

2.26% Zeodent 165 (silica abrasive)

1.00% Flavor 484 (Walmart brand)

1.00% titanium dioxide

1.00% CMC 9M31XF/Cekol 2000 (binder gum)

1.50% pure calcium

0.25% saccharin

1.50% magnesium phosphate

0.10% sodium benzoate

The foregoing components were combined as described above in Example 2.

### EXAMPLE 4

The Stearns and Ames strain of Bacillus anthracis were subjected to a battery of standard topical anti-bacterials, nutriceuticals, and herbals, including SILVADENE (generic silver sulfadiazine, vended by Hoescht Marion Roussel, now Par); SILVADENE with nystatin 0.025%; mafenide acetate, FURACIN (generic nitrofurazone, vended by Roberts), bacitracin with Polymyxin B (Poly B), silver nitrate, sodium hypochlorite (NaOCl), grapefruit seed extract (GSE), oleander extract with Aloe vera (Biotonics, San Antonio, Tex.), and a new anti-infective solution called FX (Sterifx, Inc, Shreveport, La.). Both B. anthracis strains were tested by Nathans Agar Well Diffusion Technique.

Results: The Stearns strain of B. anthracis was susceptible to all products tested except Bacitracin, Poly B and NaOCl. The most effective among the standard topicals was Bactroban® with an average inhibition zone of 45 mm, followed by mafenide acetate at 38 mm. Furacin was 33 mm with Silvadene at 19 mm. Both Silvadene with Nystatin and AgNO3 zones of inhibition were 18 mm. The nutraceuticals GSE and d-limonene had zones of inhibition of 25 mm and 30 mm, respectively, whereas the Oleander with Aloe vera had a zone size of 20 mm. The Fx product at 1× had no zone of inhibition while the 4× and 12×zones were 25 mm and 32 mm, respectively. The zones of inhibition for the more lethal and pathogenic Ames strain were comparable to those of the Stearns strain for the standard anti-infectives, nutraceuticals (i.e. GSE and d-limonene) and herbal products. Again, mafenide acetate and Bactroban® were at the top of the susceptibility list at 34 mm vs 35 mm, respectively as was the Fx 4× and 12× both at 35 mm and 46 mm, respectively. GSE and Fx product 1× zones of inhibition were both at 23 mm. d-limonene's zone of inhibition as at 21 mm. SILVADENE was at 18 mm while Nystatin/SILVADENE was 14 mm. AgNO₃ zones of inhibition was at 16 mm as was the Oleander Aloe vera product. Bacitracin, Polymyxin B and NaOCl were ineffective showing no zones of inhibition. Both strains of B. anthracis were susceptible to the standard topical antimicrobials. Bactroban®, mafenide acetate and Silvadene®. The commercial Fx product was very effective at 4× and 12× concentrations. The majority of products tested inhibited the growth of both strains of B. anthracis.

### EXAMPLE 5

Methods: Six strains of Bacillus species were tested using the Nathans Agar Well Diffusion technique in 3 replicate assays. The strain included ATCC strains of Paenibacillus polymyxa, Bacillus licheniformis, Bacillus subtilis, Bacillus sphaericus, Bacillus cereus and a wild Bacillus strain from a burn patient. The anti-infectives tested were Silvadene®, Mafenide Acetate, Furacin, Bactroban®, Bacitracin plus Polymyxin B, Silvadene® with Nystatin, 0.025% NaOCl, AgNO₃, Grapefruit Seed Extract (GSE), d-limonene, Oleander extract with Aloe vera and various concentrations of a new anti-infective solution.

Results: All anti-infectives tested were effective against all strains of Bacillus except Bacitracin with Polymyxin B where none of the strains were inhibited and NaOCl were only inhibited P. polymyxa, B. sphaericus and B. cereus with an average zone size of 16 mm. Bactroban®'s average zone of inhibition was 46 mm followed by mafenide acetate at 36 mm. Furacin was 35 mm, Silvadene was 26 mm, followed by GSE at 25 mm. Silvadene® with Nystatin was 24 mm, while Fx 1× was only effective against B. subtilis, B. sphaericus and P. polymyxa at 22 mm. Fx5x and Fx10x inhibited all Bacillus strains tested with an average zone size of 32 mm and 49 mm respectively. The Oleander extract was 18 mm while d-limonene zones were 21 mm and AgNO3 was 16 mm.

Conclusions: The standard topicals used in soft tissue wound infections could effectively eradicate cutaneous B. anthracis as would the nutriceuticals (i.e. d-limonene) and herbals tested. Moreover, the herbals and nutraeuticals could be employed effectively as aerosols in the case of inhalation anthrax, and thus, could effectively be used as therapeutic alternatives for B. anthracis infections.

### EXAMPLE 6

0.25 to 0.50 grams of each of three different toothpaste formulations (labeled C, D, and P) was applied to clinical isolates (10⁵ bacteria/ml) of gram-positive pathogens Staphylococcus aureus and Enterococcus faecalis and faecium as well as gram-negative pathogens Escherichia coli and Pseudomonas aeruginosa in accordance with the standard phenol-coefficient assay and other screening methodology for plant antimicrobial activity and incubated for 72 hrs. A 2 ml broth media was used as a positive control. The d-limonene used was purified to at least 98.5% via a distillation process. The product was purified and examined for purity via HPLC.

Formulation C comprised the toothpaste formulation described herein in Example 2.

Formulation D comprised at least 98% pure d-limonene (20%) and the remaining ingredients for Formulation C except for the calcium and magnesium (the remaining 0.5% being made up as water).

Formulation P was a placebo formulation, comprising (a) 31.175% sorbitol; (b) 25.0% ZEODENT 113; (c) 17.44% water; (d) 12.5% glycering natural kosher; (e) 6.25% Polysorbate 20; (f) 3.38% ZEODENT 165; (g) 1.25% flavoring 484 (Walmart brand); 1.25% titanium dioxide; (h) 1.25% CEKOL 2000; ( ) 0.313% sodium saccharin; and (k) 0.125% sodium benzoate.

Aliquots were subsequently cultured at 24 hours, 48 hours, and 72 hours to determine the antimicrobial effect. Appropriate media was inoculated in accordance with NCCLS standards. Blood agar was used for the gram-positive organisms, while MacConkey Agar was utilized for the gram-negative organisms.

The results of the assay are shown in Table 2, wherein all of the pathogens tested were shown to be effectively eradicated within 24 hours.

TABLE 3

| | E. coli | E. faecalis | P. aeruginosa | S. aureus |
|---|---|---|---|---|
| Formulation C | 9 mm | 14 mm | 34 mm | 12 mm |
| zone size | | | | |
| Formulation D | 14 mm | 17 mm | 28 mm | 22 mm |
| zone size | | | | |
| Formulation P | 0 mm | 0 mm | 36 mm | 10 mm |
| zone size | | | | |

### EXAMPLE 7

A mouthwash formulation was manufactured by combining the following components:

| | |
|---|---|
| Polyol | 20.0% |
| PEG 6/Ultra PEG 300 | 15.0% |
| Polysorbate 20 | 5.0% |
| d-limonene | 5.0% |
| Water | 52.7% |
| Sucralose | 0.30% |
| Belwood Wintergreen | 2.0% |

### EXAMPLE 8

A 3 week, double blind, clinical study was conducted to compare the effects on Chronic Periodontal Disease of the inventive toothpaste formulation described in Example 2 with a placebo dentifrice (i.e. without d-limonene or any other active ingredients). Male and female subjects received a scale and root plane (S/RP) and a through periodontal screening. The periodontal probing pocket depth (PD), bleeding on probing (BOP), plaque accumulation (PI), and gingival status (GI) were all measured at baseline and at 3 weeks. No professional hygiene was delivered during this study period. Mean plaque scores decreased between baseline and 3 weeks. Mean gingival scores decreased, and periodontal depth (PD) decreased slightly, but most significantly was the bleeding on probing score (BOP). It was concluded that d-limonene as an additive in a fluoride-containing dentifrice exhibited distinctive plaque inhibitors effects and decreased bleeding on probing in chronic periodontal patients.

Male and female adult subjects with a baseline Quigley-Hein Plaque Index scores of 1.5 or greater were entered into the study. All subjects received a soft-bristled toothbrush for home use and were instructed to brush their teeth twice daily (morning and evening) for 2 minutes at each tooth brushing. At the end of the 3 weeks use of their assigned dentifrice, the subjects had their teeth evaluated for plaque formulation. The results indicated that the group assigned to the d-Limonene dentifrice had less plaque formulation than the group assigned to the placebo dentifrice. All reductions in plaque formation were statistically significant at the 97% level of confidence or greater. The effect was more pronounced on the teeth that had heaviest plaque formation. These findings would appear to warrant further investigation into the potential value of the paste containing d-Limonene in inhibiting both plaque and bleeding scores in periodontal patients.

I claim:

1. A toothpaste formulation comprising d-limonene and one or more base components suitable for use as toothpaste.

2. The toothpaste formulation of claim 1, wherein said d-limonene comprises from about 10 to about 40% of said formulation.

3. The toothpaste formulation of claim 2, wherein said d-limonene comprises from about 15 to about 25% of said formulation.

4. The toothpaste formulation of claim 1, wherein said d-limonene has a purity of at least 98.5%.

5. The toothpaste formulation of claim 4, wherein said d-limonene comprises from about 10 to about 40% of said formulation.

6. The toothpaste formulation of claim 5, wherein said d-limonene comprises from about 15 to about 25% of said formulation.

7. The toothpaste formulation of claim 1, further including calcium and a magnesium salt.

8. The toothpaste formulation of claim 4, further including calcium and a magnesium salt.

9. A toothpaste formulation comprising d-limonene, a polyol, water, silica, glycerin, a surfactant, and a binder.

10. The toothpaste formulation of claim 9, wherein said d-limonene has a purity of at least 98.5%.

11. The toothpaste formulation of claim 9, wherein said d-limonene comprises from about 10 to about 40% of said formulation.

12. The toothpaste formulation of claim 11, wherein said d-limonene comprises from about 15 to about 25% of said formulation.

13. A mouthwash formulation comprising d-limonene, water, a surfactant, a polyol, and a flavoring agent.

14. The mouthwash formulation of claim 13, wherein said d-limonene has a purity of at least 98.5%.

15. The mouthwash formulation of claim 14, wherein said d-limonene comprises from about 5 to about 15% of said formulation.

16. A method for inhibiting the growth or killing bacteria within the oral cavity of an animal, said method comprising administering a therapeutically effective amount of a formulation comprising d-limonene within the oral cavity for a time sufficient to effectively eradicate said bacteria.

17. The method of claim 16, wherein said d-limonene has a purity of at least 98.5%.

18. The method of claim 1, wherein said bacteria are selected from the group of Porphyromonas gingivalis, Strep. mutans, Strep. pyogenes, Bacteroides species, Actinobacillus action mycetemcomitons, Prevotella intermedia, Fusobacterium nucleatum, Campylobacter rectus, Eikenella corrodens, Peptostreptoloccus micros, Selenomonas sp., Eubacterium sp., Streptococcus species, Spirochetes treponema denticola, and Treponema pallidum.

19. The method of claim 18, wherein said d-limonene has a purity of at least 98.5%.

20. A method for treating periodontal disease and preventing tooth decay, said method comprising applying an effective amount of the toothpaste formulation recited in claim 1 to an animal's teeth and gums for a time sufficient to remove, kill, or inhibit the growth of pathogens responsible causing said periodontal disease and tooth decay.

21. The method of claim 20, wherein said toothpaste formulation comprises from about 10% to about 40% of d-limonene.

22. The method of claim 21, wherein said d-limonene has a purity of at least 98.5%.

23. The method of claim 20, wherein said toothpaste formulation comprises from about 15% to about 25% of d-limonene.

24. The method of claim 23, wherein said d-limonene has a purity of at least 98.5%.
